(19) United States
(12) Patent Application Publication
(10) Pub. No.: US 2025/0249175 A1
(43) Pub. Date: Aug. 7, 2025

(54) DRUG DELIVERY SYSTEM

(71) Applicant: Analog Devices, Inc., Wilmington, MA (US)

(72) Inventors: Vikram Venkatadri, Ayer, MA (US); David Frank Bolognia, Charlestown, MA (US)

(21) Appl. No.: 18/431,836

(22) Filed: Feb. 2, 2024

(51) Int. Cl. A61M 5/172

(52) U.S. Cl. CPC A61M 5/172; A61M 2205/50

(57) ABSTRACT

A sensor module may include a housing comprising a channel extending through a longitudinal axis of the housing. The channel is configured to receive a fluid substance. The sensor module includes an outlet valve in fluid communication with the channel and a capacitive sensor positioned inside the channel. The sensor module can include an actuator at least partially surrounded by the capacitive sensor, and a pump including a fluid reservoir configured to contain a solution. During operation of the sensor module, activation of the pump pushes at least some of the solution from the fluid reservoir into the channel to advance the actuator into the chamber.

### CROSS-REFERENCE TO RELATED APPLICATIONS

Any and all applications for which a foreign or domestic priority claim is identified in the Application Data Sheet as filed with the present application are hereby incorporated by reference under 37 CFR 1.57.

### BACKGROUND

### Field

The field relates to fluid delivery systems and, in particular, to drug delivery systems.

### Description of the Related Art

Fluid delivery systems can deliver a substance, e.g., a fluid substance, from one location to another. For example, a substance delivery system can include a drug delivery system. In a drug delivery system, a drug can be stored in one place (e.g., a pod) and the drug can be delivered to a patient's body. It can be important to accurately determine the amount (e.g., volume) of drugs being delivered to a patient.

### SUMMARY

In some aspects, the techniques described herein relate to a sensor module. The sensor module can include a housing including a channel extending through a longitudinal axis of the housing, wherein the channel is configured to receive a fluid substance; an outlet valve in fluid communication with the channel; a capacitive sensor positioned inside the channel; an actuator at least partially surrounded by the capacitive sensor; and a pump including a fluid reservoir configured to contain a solution, wherein during operation of the sensor module, activation of the pump pushes at least some of the solution from the fluid reservoir into the channel to advance the actuator into the channel.

The sensor module of any of the preceding paragraphs and/or any of the sensor modules disclosed herein can include one or more of the following features. In some cases, during operation of the sensor module, advancement of the actuator into the channel can eject at least a portion of the fluid substance in the channel via the outlet valve. In some aspects, the capacitive sensor can be configured to measure a capacitance across the channel. The capacitance across the channel can provide an indication of an amount of the fluid substance ejected via the outlet valve. In some cases, the pod can be configured to store the fluid substance. In some aspects, the capacitive sensor can include a first capacitor plate and a second capacitor plate. The outlet valve can be fluidly connected to a needle assembly configured to deliver the fluid substance ejected via the outlet valve to a patient. In some cases, the pump can include an electroosmotic pump. In some aspects, the channel can define a first chamber portion and a second chamber portion, the capacitive sensor positioned inside the first chamber portion, and wherein the second chamber portion is configured to receive the fluid substance. In some cases, at least a portion of the first chamber portion and at least a portion of the second chamber portion can be separated by the actuator. In some aspects, the capacitive sensor can be exposed to the solution when the solution is in the channel.

In some variants, the techniques described herein relate to a sensor module. The sensor module can include a housing including a channel configured to receive a fluid substance; an inlet valve in fluid communication with the channel and removably connected to a pod containing the fluid substance; a capacitive sensor positioned inside the channel; and an actuator, wherein during operation of the sensor module, reciprocation of the actuator along the channel ejects at least a portion of the fluid substance in the channel via an outlet valve.

The sensor module of any of the preceding paragraphs and/or any of the sensor modules disclosed herein can include one or more of the following features. In some cases, the capacitive sensor includes a first capacitor plate and a second capacitor plate. In some aspects, the sensor module can include a controller in communication with the capacitive sensor, the controller configured to measure a capacitance across the first and second capacitor plates and, based on the measured capacitance, determine an amount of the fluid substance ejected via the outlet valve. The sensor module can include a pump, wherein activation of the pump can be configured to reciprocate the actuator along the channel. In some cases, the pump can include an electroosmotic pump, the electroosmotic pump in communication with a fluid reservoir containing a solution, wherein, during operation of the sensor module, activation of the electroosmotic pump pushes at least some of the solution from the fluid reservoir into the channel, thereby pushing the actuator away from the electroosmotic pump. In some aspects, the outlet valve can be fluidly connected to a needle assembly configured to deliver the fluid substance ejected via the outlet valve to a patient.

In some variants, the techniques described herein relate to a sensor module. The sensor module can include a housing including a channel extending through a longitudinal axis of the housing, the channel defining a first chamber and a second chamber; an outlet valve in fluid communication with the channel; and an inlet valve in fluid communication with the channel; wherein the second chamber is configured to receive a fluid substance via the inlet valve; a capacitive sensor including a first capacitor plate and a second capacitor plate, wherein the first and second capacitor plates are positioned inside the first chamber; an actuator at least partially surrounded by the first and second capacitor plates; a controller in communication with the capacitive sensor; and a pump, wherein activation of the pump is configured to reciprocate the actuator along the channel; wherein, during operation of the sensor module, reciprocation of the actuator along the channel ejects at least a portion of the fluid substance in the second chamber via the outlet valve; wherein the controller is configured to measure a capacitance across the first and second capacitor plates and, based on the measured capacitance, determine an amount of the fluid substance ejected via the outlet valve.

The sensor module of any of the preceding paragraphs and/or any of the sensor modules disclosed herein can include one or more of the following features. In some cases, the inlet valve can be removably connected to a pod configured to store the fluid substance. In some aspects, the outlet valve can be fluidly connected to a needle assembly configured to deliver the fluid substance ejected via the outlet valve to a patient. The pump can include an electroosmotic pump, the electroosmotic pump in communication with a fluid reservoir containing a solution, wherein, during operation of the sensor module, activation of the electroosmotic pump pushes at least some of the solution from the fluid reservoir into the first chamber, thereby pushing at least a portion of the actuator into the second chamber. In some cases, the first and second capacitor plates can be at least partially exposed to the solution when the electroosmotic pump is activated. In some aspects, the electronics assembly can include a first plurality of pins connecting the electronics assembly to the first capacitive plate, and a second plurality of pins connecting the electronics assembly to the second capacitive plate. The controller can be integrated into the electronics assembly.

### BRIEF DESCRIPTION OF THE DRAWINGS

Various implementations will be described hereinafter with reference to the accompanying drawings. There implementations are illustrated and described by example only and are not intended to limit the scope of the disclosure. In the drawings, similar elements have similar reference numerals.

FIG. 1 is a block diagram of a drug delivery system according to one embodiment.

FIG. 2A illustrates a top perspective view of a drug delivery system.

FIG. 2B illustrates a bottom perspective view of the drug delivery system shown in FIG. 2A.

FIG. 2C illustrates the interior of the drug delivery system shown in FIGS. 2A and 2B.

FIG. 2D illustrates the drug delivery system shown in FIGS. 2A-2C with a drug pod detached from the system.

FIG. 3A illustrates a semi-transparent view of a sensor module for a drug delivery system, according to one embodiment.

FIG. 3B illustrates a front perspective view of the sensor module for a drug delivery system shown in FIG. 3A, according to one embodiment.

FIG. 3C illustrates a rear perspective view of the sensor module for a drug delivery system shown in FIG. 3A, according to one embodiment.

FIGS. 4A and 4B illustrate a housing for a sensor module according to one embodiment.

FIGS. 5A-5C illustrate a sensor module with the housing detached from the sensor module.

FIGS. 6A-6C illustrate cross-section views of a sensor module and the movement of an actuator inside the sensor module.

### DETAILED DESCRIPTION

Drug delivery systems can be used to deliver a fluid substance (e.g., a drug) disposed in a reservoir to a target location (e.g., inside a patient's vasculature, into analysis equipment, or to any other suitable target location). The drug can include, for example, insulin for treating diabetes, an anti-nausea drug for chemotherapy, etc. The drug delivery system can include a flow control device (e.g., a pump assembly) and/or a drug delivery device (e.g., a drug pod and/or a needle assembly).

The flow control device can include a pump assembly having a pump, a flow meter, a valve, and an electronics unit. The flow meter can monitor/measure a flow rate and/or a flow amount (e.g., volume) of the substance that has already been delivered and/or that will be delivered. The flow meter can manage the flow rate and a volume of the substance to deliver to the target location. As one example, it can be important to accurately and repeatedly monitor an amount of insulin delivered to a diabetic patient and to control the timing of the delivery.

The pump assembly can include a check valve. The valve can include any valve but, in some cases, may include a shutoff valve and/or a check valve. A shutoff valve can open to permit flow of the fluid substance and close to shut of the flow of the fluid substance. A check valve can prevent or mitigate a backflow of the fluid substance. The flow meter can be in communication with the valve to manage the operation of the valve. For example, the electronics unit of the pump assembly can include a controller in electrical communication with the valve and the flow meter. The controller can send instructions to the valve to open and/or close the valve based on signals received from the flow meter. The valve can include a gate that can open and/or close to control the flow of the substance. The valve can include a mechanical actuator that can cause the gate to open and/or close.

In some cases, the pump can comprise an electro-osmatic (EO) pump. The pump can include an expandable or deformable chamber configured to receive and contain a solution (e.g., an electrolyte) therein. The expandable or deformable chamber can include an elastic or expandable diaphragm that can expand in response to a pressure difference in the chamber caused by a movement or a volumetric expansion of the solution in the chamber. The solution can comprise any suitable solution. For example, the solution can comprise reverse osmosis (RO) water. The expansion of the diaphragm can actuate the gate to open/close. The gate can have a closed state in which the gate is completely closed. The gate can have an opened state. The opened state can comprise a fully opened state in which the gate is fully/completely opened. The opened state can comprise an intermediate state in which the gate is partially opened. The intermediate state can be a state in between the closed state and the fully opened state, which can enable a non-zero flow through the valve that is less than the flow rate in the opened state.

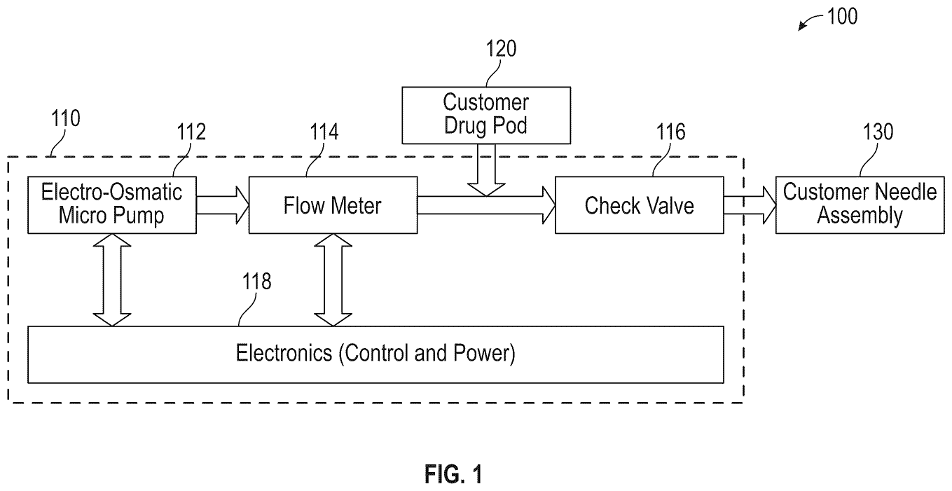

FIG. 1 illustrates a block diagram of a drug delivery system according to the disclosure herein. The drug delivery system 100 can include a pump assembly 110, a drug pod 120, and a needle assembly 130. The pump assembly 110, the drug pod 120, and the needle assembly 130 can be in fluid communication with and removably coupled to each other. In some cases, the pump assembly 110, the drug pod 120, and the needle assembly 130 can all be part of a single assembly, such as contained within a housing and/or secured to the housing.

The pump assembly 110 can include a pump 112, a flow meter 114, a valve 116, and/or an electronics assembly 118. The pump 112 can include any type of pump but in some cases may include an electro-osmatic micro pump. Additional details about electro-osmatic micro pumps can be found on U.S. Pub. No. 2022/0062535 A1, titled FLUID DELIVERY SYSTEM, which is hereby incorporated herein in its entirety. The pump 112 can move the fluid from the drug pod 120 to a target location via the needle assembly 130. For example, in operation, the pump 112 can move the fluid from the drug pod 120 to the pump assembly 110 and into the vasculature of a patient via the needle assembly 130.

The flow meter 114 can monitor a flow rate and/or a flow amount of the substance flowing through the flow meter 114 from the drug pod 120. In some cases, the flow meter 114 can monitor a flow of a fluid substance to measure a delivered volume of the substance, a flow rate, and/or a flow direction (e.g., forward flow or backflow). The measured data can be compared against a predetermined or prescribed dosage of the substance. The measured data can be used to operate, for example, the pump 112 and/or the valve 116. The drug pod 120 can include a container for receiving and storing a fluid substance (e.g., drug, insulin, etc.).

The needle assembly 130 can comprise a conduit (e.g., a tube) and a needle that is coupled to the conduit. In some applications, the needle can be inserted into a patient's body through the skin such that the fluid substance (e.g., drug) delivered from the drug pod 120 via the pump assembly 110 is delivered to a vasculature of the patient through the needle assembly 130.

The electronics assembly 118 can control operation of the pump assembly 110. The electronics assembly 118 can include processing electronics that can be programmed to control operations of the pump assembly 110. A controller of the electronics assembly 118 can include one or more processors, and/or one or more memory devices, etc. In some cases, the pump 112, the flow meter 114, and/or the valve 116 can connect to the controller of the electronics assembly 118, and can be controlled by the controller. In some embodiments, the pump 112, the flow meter 114, and/or the valve 116 can be connected to the controller of the electronics assembly 118 through a wired connection, or wirelessly. The controller can include or be connected to a user interface that can allow control of the pump assembly 110, or to monitor the status of the pump assembly 110. In some cases, the controller can be connected to other sensors, such as an accelerometer, a thermometer, etc. The controller can be connected to a vital sign monitoring device. In some cases, the controller can be programmed to deliver a predefined amount (e.g., 1 ml, 2 ml, 3 ml, 4 ml, 5 ml, etc.) of a substance (e.g., a drug) to a target location over time (e.g., every 1 hour, 2 hours, 6 hours, 12 hours, 24 hours, etc.).

The one or more memory devices of the electronics assembly 118 can store one or more instructions that, when executed by the controller of the electronics assembly 118, can control operations of the pump assembly 110. For instance, the one or more instructions can include instructions to activate and/or deactivate the pump 112, activate and/or deactivate the flow meter 114, open and/or close the valve 116, deliver the substance from the drug pod 120 to the pump assembly 110 and/or the patient via the needle assembly 130.

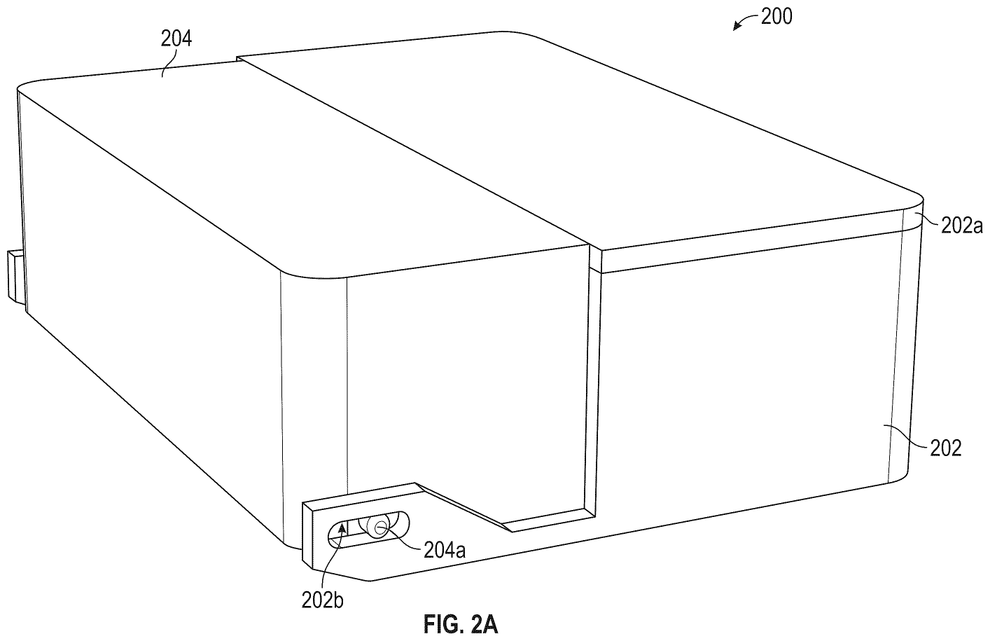

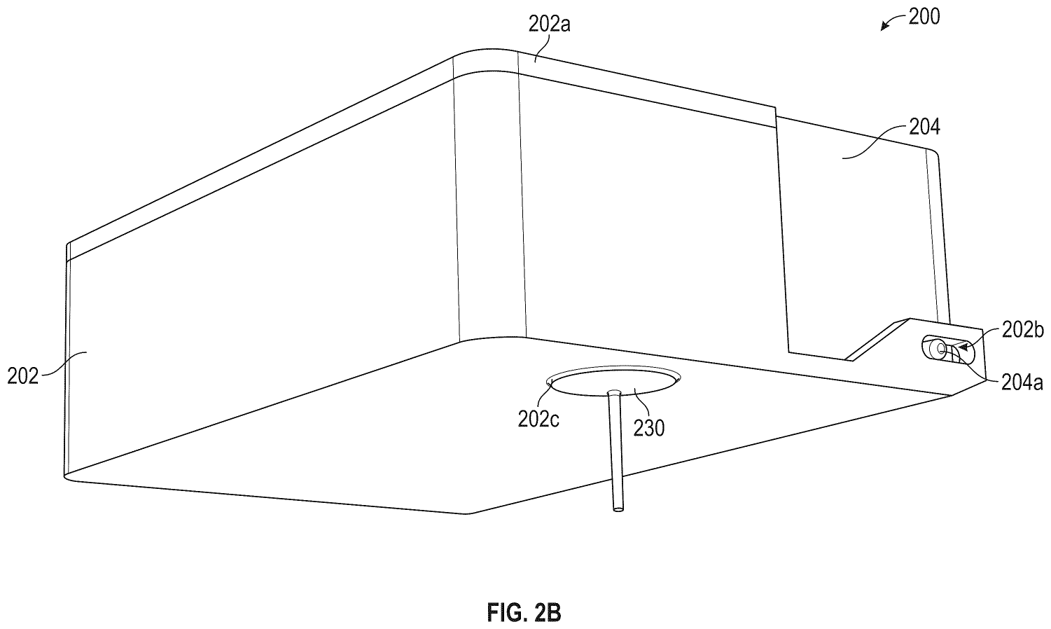

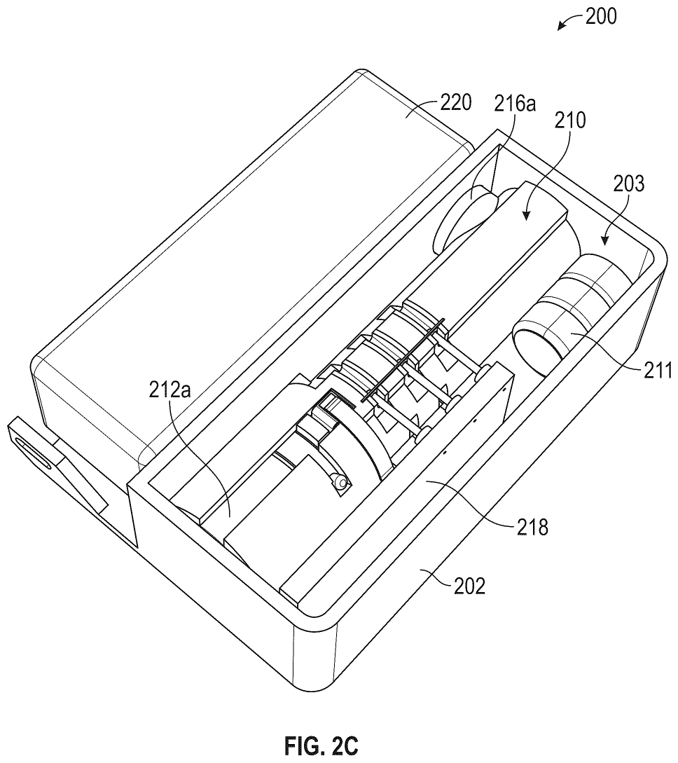

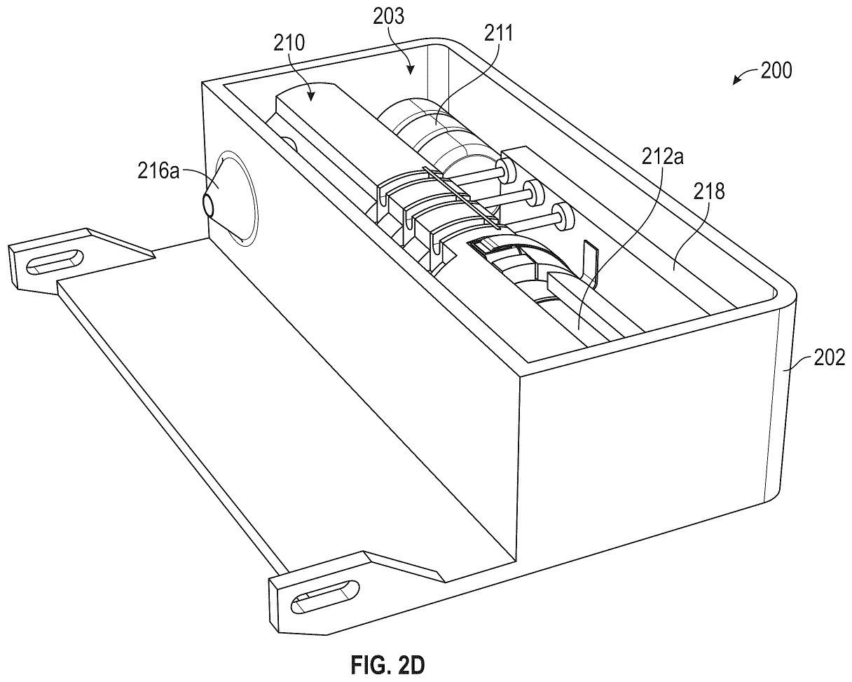

FIGS. 2A-2D illustrate a drug delivery system 200 according to one embodiment. The drug delivery system 200 can include a main housing 202 and a reservoir housing 204, as shown in FIG. 2A. In some cases, the main housing 202 can include a lid 202a. The reservoir housing 204 can be removably connected to the main housing 202. For example, the reservoir housing 204 can include one or more projections 204a that can be received by one or more slots 202b of the main housing 202. The slots 202b can receive and secure the projections 204a thereby preventing the reservoir housing 204 from accidentally detaching from the main housing 202.

As shown in FIG. 2B., the main housing 202 can include a hole 202c along a bottom portion of the main housing 202. At least a portion of a needle assembly 230 can extend through the hole 202c. In some cases, the needle assembly 230 can be sealed to the hole 202c of the main housing 202 to protect the interior of the main housing 202 from the outside environs and/or inhibit leaks through the hole 202c.

FIG. 2C shows the main housing 202 with the lid 202a removed from the main housing 202. The main housing 202 can define a chamber 203. The chamber 203 can receive and secure a pump assembly 210 (also referred herein as a sensor module). In some cases, the pump assembly 210 can be in fluid communication with the reservoir 220 (e.g., drug pod). The reservoir 220 can receive and store a fluid substance (e.g., drug, insulin, etc.). In some cases, the reservoir 220 can be positioned inside the reservoir housing 204, which is detached from the main housing 202 in FIG. 2C. The pump assembly 210 and the reservoir 220 can be in fluid communication with each other via an inlet valve 216a of the pump assembly 210, as shown in in FIG. 2D. The reservoir 220 can beneficially be replaced with a different reservoir 220 when the substance contained within reservoir 220 is depleted.

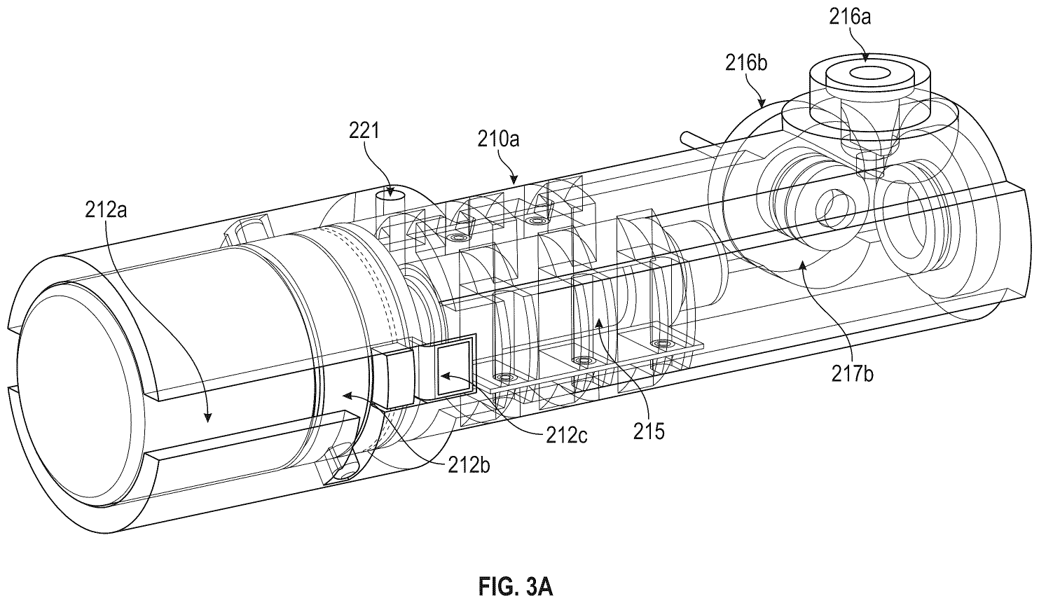

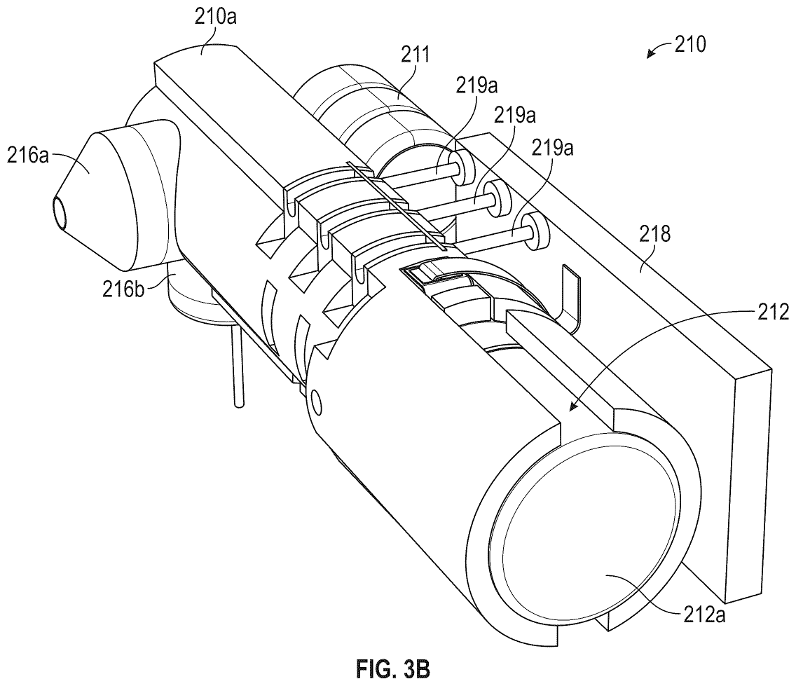

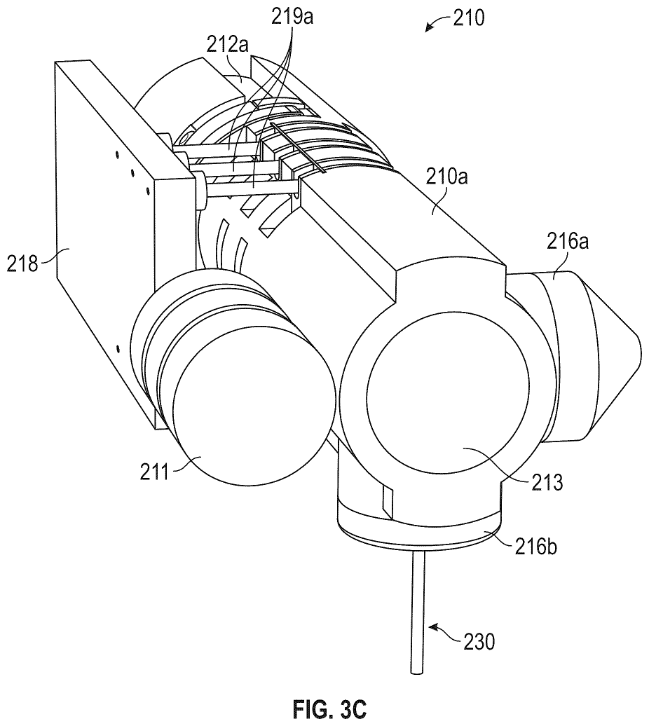

FIGS. 3A-3C show different views of the pump assembly 210. The pump assembly 210 can include a housing 210a (e.g., a vessel), a pump 212, an inlet valve 216a, an outlet valve 216b, and an electronics assembly 218. In some cases, the housing 210a can receive and secure at least some of the components of the pump assembly 210. For instance, the housing 210a can receive and/or secure the pump 212, the inlet valve 216a, and/or the outlet valve 216b. As will be further described below, the electronics assembly 218 can be secured to the housing 210a via one or more pins (e.g., a first plurality of pins 219a and/or a second plurality of pins 219b). In some cases, the pump assembly 210 can include one or more batteries 211. The pump assembly 210 can be connected to the batteries 211 which can power the pump assembly 210 and its components (e.g., the electronics assembly 218, the pump 212, the inlet valve 216a, the outlet valve 216b, etc.). The pump 212 can include a fluid reservoir 212a. As will be described in further detail below, the fluid reservoir 212a can receive and contain a solution (e.g., an electrolyte) therein. In some cases, the fluid reservoir 212a can be fixedly coupled to the housing 210a via a compression collar 212b. The compression collar 212b can be positioned inside a channel 217 of the housing 210a.

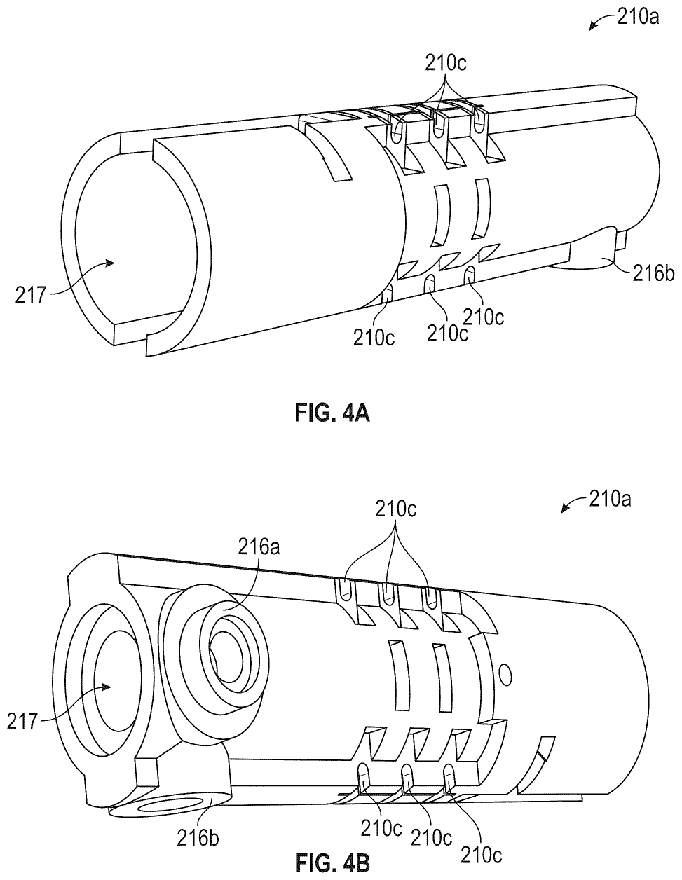

As shown in FIGS. 4A-4B the housing 210a can include a channel 217 extending through a longitudinal axis of the housing 210a. The housing 210a can also include a plurality of slots 210c. The plurality of slots 210c can allow communication between the channel 217 and the exterior of the housing 210a. This can beneficially allow components positioned outside the housing 210a (e.g., the first plurality of pins 219a and/or the second plurality of pins 219b) to communicate (e.g., be connected) with components positioned inside the channel 217. The fluid reservoir 212a of the pump 212 can be positioned inside the housing 210a. For example, the fluid reservoir 212a can be positioned in the channel 217. In some cases, at least one end of the channel 217 can be sealed. For instance, a cap 213 can be positioned on and secured to one end of the housing 210a, as shown in FIG. 3C. The cap 213 can be sealed to the housing 210a to close one end of the channel 217. This can beneficially prevent leaks through one end of the channel 217.

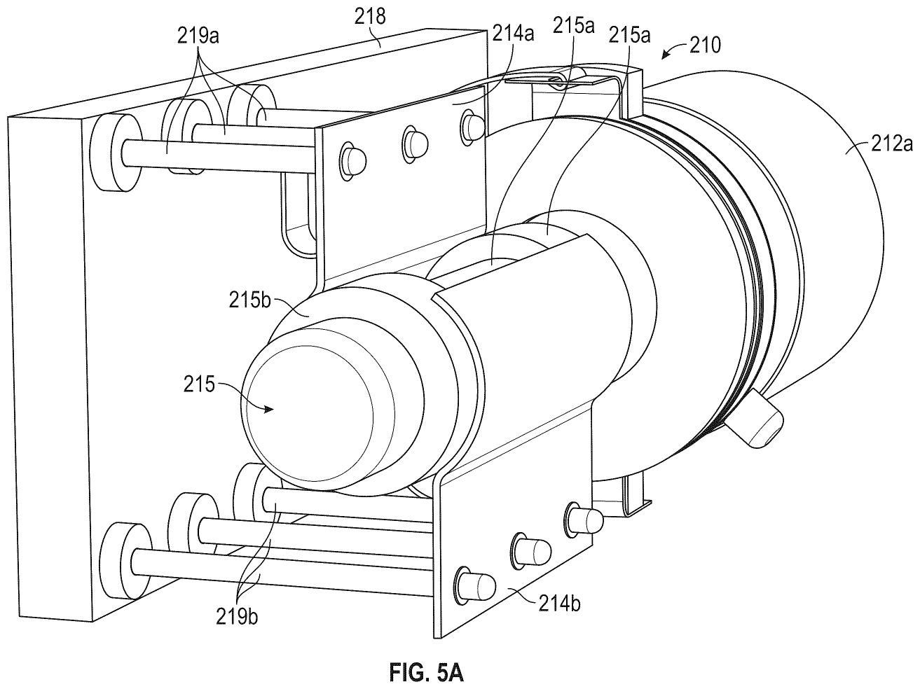

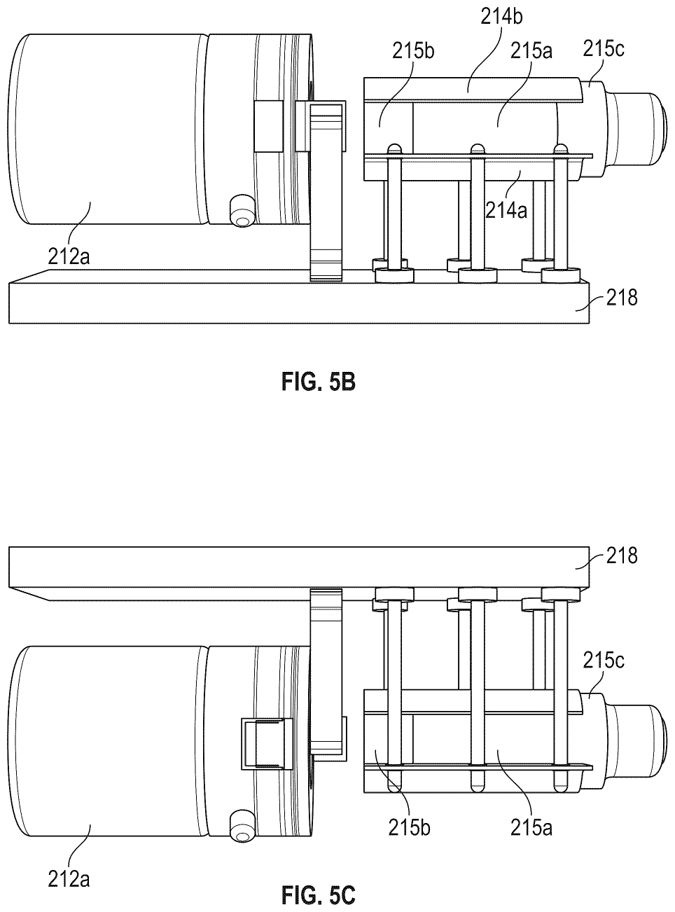

FIGS. 5A-5C show the pump assembly 210 with the housing 210a removed. In some cases, the electronics assembly 218 can be connected to one or more capacitor plates. Each of the one or more capacitor plates can include an electrode. The electronics assembly 218 can be connected to a first capacitor plate 214a via the first plurality of pins 219a, and/or to second capacitor plate 214b via the second plurality of pins 219b. Although reference is made to the first and second capacitor plates 214a, 214b being connected to the electronics assembly via a first plurality of pins 219a and a second plurality of pins 219b respectively, the electronics assembly 218 can be connected to the capacitor plates 214a, 214b via a single set of pins. In some cases, the electronics assembly 218 is in communication with the capacitor plates 214a, 214b wirelessly. The first capacitor plate 214a and the second capacitor plate 214b can be positioned inside the channel 217 of the housing 210a.

As shown in FIG. 5A, the first capacitor plate 214a and the second capacitor plate 214b can partially surround an actuator 215. In some cases, the actuator 215 can include a dumbbell shape. That is, the actuator 215 can include a bar 215a, a first round end 215b, and a second round end 215c. The actuator 215 can be mechanically coupled to the pump 212. In operation, the pump 212 can translate the actuator back and forth along the channel 217. As further explained below, the translation of the actuator 215 along the channel 217 can cause fluid inside the channel 217 to be ejected from the pump assembly 210 via the outlet valve 216b and the needle assembly 230.

In some cases, the electronics assembly 218 can include a controller. The controller can control operation of the pump assembly 210. The controller can include processing electronics that are programmed to control operations of the pump assembly 210. The controller can include one or more processors, and/or one or more memory devices, etc. For example, the pump assembly 210, the first capacitor plate 214a, the second capacitor plate 214b, the inlet valve 216a, and/or or the outlet valve 216b can connect to the controller, and can be controlled by the controller. In some cases, the controller can be connected to one or more sensors, such as an accelerometer, thermometer, etc.

The controller of the electronics assembly 218 can be programmed to send a start signal to the pump 212 to activate the pump at a predetermined time, on command from the user or clinician, or based on other criteria. In response to the signal, the pump assembly 210 can drive a substance from the reservoir 220 through a flow path by way of the inlet valve 216a, the channel 217, the outlet valve 216b, and the needle assembly 230 to a target location. The controller can transmit a valve open signal to open the inlet valve 216a so as to allow the substance to flow from the reservoir 220 into the channel 217 of the housing 210a. The controller can also transmit a valve close signal to close the inlet valve 216a once the substance has flowed from the from the reservoir 220 into the channel 217. To eject the substance from the channel 217 into the target location via the needle assembly 230, the controller of the electronics assembly can activate the pump 212. Activation of the pump 212 can cause translation of the actuator 215 along the longitudinal axis of the housing 210a. Translation of the actuator 215 can force the substance out of the channel 217 via the outlet valve 216b and the needle assembly 230. To allow the substance to exit the housing 210a via the needle assembly 230, the controller of the electronics assembly 218 can transmit a valve open signal to the outlet valve 216b so as to allow the substance to flow through the outlet valve 216b. To prevent backflow of the substance into the channel 217, the controller of the electronics assembly 218 can transmit a valve close signal to the outlet valve 216b so as to prevent the substance flow flowing back into the housing 210a.

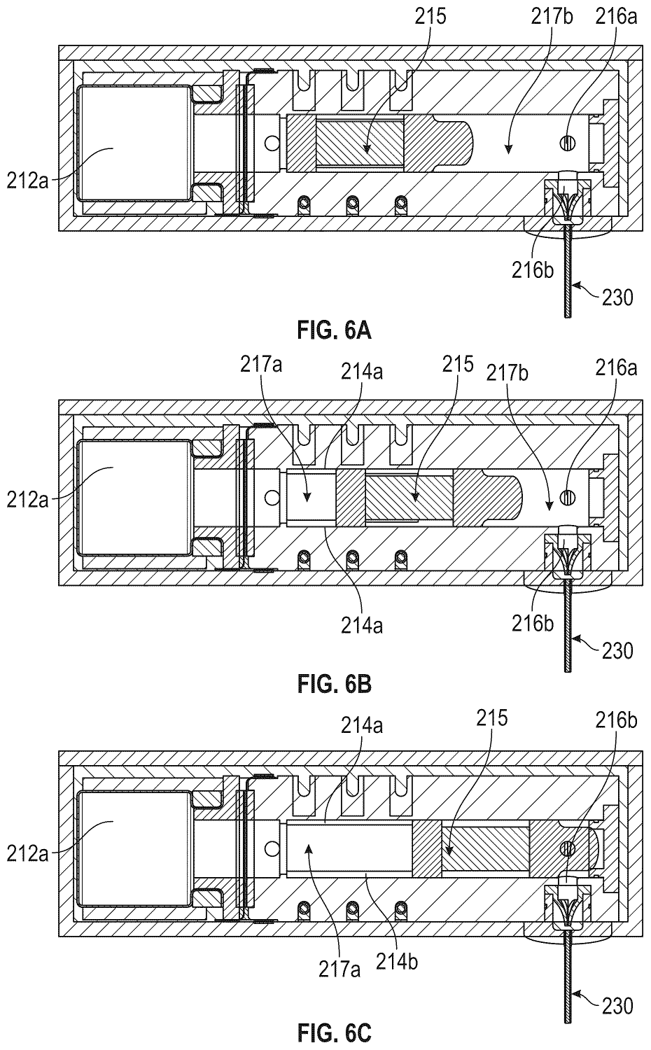

The pump 212 can comprise an electro-osmatic (EO) pump. The pump can include a fluid reservoir 212a, as shown in FIGS. 6A-6C, configured to receive and contain a solution (e.g., an electrolyte) therein. The fluid reservoir 212a can include an elastic or expandable diaphragm that can expand in response to a pressure difference in the chamber caused by a movement or a volumetric expansion of the solution in the chamber. The solution can comprise any suitable solution. For example, the solution can comprise reverse osmosis (RO) water.

The pump 212 can transition at least from a first state to second state in response to an electric field being applied to the pump 212. In some cases, the electric field can be applied to the pump 212 via one or more contacts 212c. The contacts 212c can be in communication with the batteries 211 and the pump 212. In the first state, as shown in FIG. 6A, the solution of the pump 212 can be fully contained within the fluid reservoir 212a. When an electric field is applied, the pump 212 can transition from the first state to the second state. In the second state, the pressure generated by the pump 212 can push at least a portion of the fluid contained within the fluid reservoir 212a out of the fluid reservoir 212a and into the channel 217 of the housing 210a. The fluid being pushed out of the fluid reservoir 212a into the channel 217 can cause the actuator 215 to translate from a starting position, as shown in FIG. 6A, along the channel 217, to a second potion, as shown in FIG. 6C. When the electric field is interrupted, the fluid inside the channel 217 can return to the fluid reservoir 212a thereby causing the actuator 215 to return to the starting position.

In some cases, the channel 217 can define a first chamber 217a and a second chamber 217b, as shown in FIGS. 6A-6C. The first chamber 217a can be in fluid communication with an air purge line 221. The air purge line 221 can include an opening in the housing 210a and be in fluid communication with the outside environs and/or the chamber 203. The air purge line 221 can beneficially allow air to be removed from the first chamber 217a. In some cases, air can be introduced into the first chamber 217a via the air purge line 221. The fluid being pushed from the fluid reservoir 212a can be pushed into the first chamber 217a of the channel 217. In some cases, the substance (e.g., a drug) contained within the reservoir 220 can be pushed into the second chamber 217b of the channel 217. The substance contained within the reservoir 220 can be pushed into the second chamber 217b when the pump 212 is in the first state. After pushing the substance into the second chamber 217b from the reservoir 220 via the inlet valve 216a, the inlet valve 216a can be closed to prevent the substance from flowing back into the reservoir 220. As the pump 212 transitions from the first state to the second state, the actuator 215 can push the substance out of the second chamber 217b via the outlet valve 216b and the needle assembly 230.

The first capacitor plate 214a and the second capacitor plate 214b can be exposed to the channel 217. For example, as shown in FIGS. 6B and 6C, at least a portion of the first and second capacitor plates 214a and 214b can be positioned inside the first chamber 217a of the channel 217. The first and second capacitor plates 214a and 214b can be in direct contact with the fluid from the fluid reservoir 212a when the fluid is pushed into the first chamber 217a of the channel 217. In some cases, the first and second capacitor plates 214a and 214b can be configured to measure or monitor a flow rate and/or flow amount of a fluid substance (e.g., a drug) that flows through the channel 217 and/or that is pushed out of the channel 217 via the outlet valve 216b. The ability of the first and second capacitor plates 214a and 214b to contact the fluid from the fluid reservoir 212a can beneficially allow for more accurate flow rate and/or flow metering.

As the fluid from the fluid reservoir 212a is pushed into the channel 217, the fluid can contact the first and/or second capacitor plates 214a and 214b. An amount of energy (e.g., capacitance) stored by the capacitor plates 214a and 214b can change depending on the volume of fluid inside the channel 217. The controller of the electronics assembly 218, which can be coupled to the first and second capacitor plates 214a and 214b via the first and second plurality of pins 219a and 219b respectively, can use the differences in the amount of energy stored by the first and second capacitor plates to determine the flow rate and/or volume of the fluid inside the first chamber 217a.

A volume of the substance (e.g., drug) evacuated from the channel 217 via the outlet valve 216b can be determined based on the volume of the fluid reservoir inside the first chamber 217a as measured by the first and second capacitor plates 214a and 214b. For example, the volume of substance (e.g., drug) evacuated from the channel 217 can be about the same, the same, and/or proportionate to the volume of fluid in the first chamber 217a as measured by the first and second capacitor plates 214a and 214b and/or the electronics assembly 218. For instance, a volume measurement (e.g., 1 ml, 2 ml, 3 ml, etc.) of the fluid inside the first chamber 217a can indicate that the same volume of the substance (e.g., the drug) has been evacuated (e.g., delivered to a target location via the outlet valve 216b and/or the needle assembly 230) from the channel 217. In some cases, a measurement of the volume of the fluid inside the first chamber 217a can indicate that a proportionate volume of substance (e.g., drug) has been evacuated from the channel 217. For example, a volume of 1 ml of fluid inside the first chamber 217a may indicate that 2 ml of substance (e.g., drug) have been evacuated from the channel 217. As another example, a volume of 1 ml of fluid inside the first chamber 217a may indicate that. 5 ml of substance (e.g., drug) have been evacuated from the channel 217. Although ratios of 2:1 and 1:2 were described in the example above, any ratio can be used (e.g., 1:3; 1:4; 1:5; 1:10; 3:1; 4:1; 10;1, etc.).

Although this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. A sensor module comprising:
a housing comprising a channel extending through a longitudinal axis of the housing, wherein the channel is configured to receive a fluid substance;
an outlet valve in fluid communication with the channel;
a capacitive sensor positioned inside the channel;
an actuator at least partially surrounded by the capacitive sensor; and
a pump comprising a fluid reservoir configured to contain a solution, wherein during operation of the sensor module, activation of the pump pushes at least some of the solution from the fluid reservoir into the channel to advance the actuator into the channel.

2. The sensor module of claim 1, wherein, during operation of the sensor module, advancement of the actuator into the channel ejects at least a portion of the fluid substance in the channel via the outlet valve.

3. The sensor module of claim 1, wherein the capacitive sensor is configured to measure a capacitance across the channel.

4. The sensor module of claim 3, wherein the capacitance across the channel provides an indication of an amount of the fluid substance ejected via the outlet valve.

5. The sensor module of claim 1, further comprising an inlet valve in fluid communication with the channel and a pod, the pod is configured to store the fluid substance.

6. The sensor module of claim 1, wherein the capacitive sensor comprises a first capacitor plate and a second capacitor plate.

7. The sensor module of claim 1, wherein the outlet valve is fluidly connected to a needle assembly configured to deliver the fluid substance ejected via the outlet valve to a patient.

8. The sensor module of claim 1, wherein the pump comprises an electroosmotic pump.

9. The sensor module of claim 1, wherein the channel defines a first chamber portion and a second chamber portion, the capacitive sensor positioned inside the first chamber portion, and wherein the second chamber portion is configured to receive the fluid substance.

10. The sensor module of claim 9, wherein at least a portion of the first chamber portion and at least a portion of the second chamber portion are separated by the actuator.

11. The sensor module of claim 1, wherein the capacitive sensor is exposed to the solution when the solution is in the channel.

12. A sensor module comprising:
a housing comprising a channel configured to receive a fluid substance;
an inlet valve in fluid communication with the channel and removably connected to a pod containing the fluid substance;
a capacitive sensor positioned inside the channel; and
an actuator, wherein during operation of the sensor module, reciprocation of the actuator along the channel ejects at least a portion of the fluid substance in the channel via an outlet valve.

13. The sensor module of claim 12, wherein the capacitive sensor comprises a first capacitor plate and a second capacitor plate.

14. The sensor module of claim 13, further comprising a controller in communication with the capacitive sensor, the controller configured to measure a capacitance across the first and second capacitor plates and, based on the measured capacitance, determine an amount of the fluid substance ejected via the outlet valve.

15. The sensor module of claim 12, further comprising a pump, wherein activation of the pump is configured to reciprocate the actuator along the channel.

16. The sensor module of claim 15, wherein the pump comprises an electroosmotic pump, the electroosmotic pump in communication with a fluid reservoir containing a solution, wherein, during operation of the sensor module, activation of the electroosmotic pump pushes at least some of the solution from the fluid reservoir into the channel, thereby pushing the actuator away from the electroosmotic pump.

17. The sensor module of claim 12, wherein the outlet valve is fluidly connected to a needle assembly configured to deliver the fluid substance ejected via the outlet valve to a patient.

18. A sensor module comprising:
a housing comprising:
a channel extending through a longitudinal axis of the housing, the channel defining a first chamber and a second chamber;
an outlet valve in fluid communication with the channel; and
an inlet valve in fluid communication with the channel;
wherein the second chamber is configured to receive a fluid substance via the inlet valve;
a capacitive sensor comprising a first capacitor plate and a second capacitor plate, wherein the first and second capacitor plates are positioned inside the first chamber;
an actuator at least partially surrounded by the first and second capacitor plates;
a controller in communication with the capacitive sensor; and
a pump, wherein activation of the pump is configured to reciprocate the actuator along the channel;
wherein, during operation of the sensor module, reciprocation of the actuator along the channel ejects at least a portion of the fluid substance in the second chamber via the outlet valve;
wherein the controller is configured to measure a capacitance across the first and second capacitor plates and, based on the measured capacitance, determine an amount of the fluid substance ejected via the outlet valve.

19. The sensor module of claim 18, wherein the inlet valve is removably connected to a pod configured to store the fluid substance.

20. The sensor module of claim 18, wherein the outlet valve is fluidly connected to a needle assembly configured to deliver the fluid substance ejected via the outlet valve to a patient.

21. The sensor module of claim 18, wherein the pump comprises an electroosmotic pump, the electroosmotic pump in communication with a fluid reservoir containing a solution, wherein, during operation of the sensor module, activation of the electroosmotic pump pushes at least some of the solution from the fluid reservoir into the first chamber, thereby pushing at least a portion of the actuator into the second chamber.

22. The sensor module of claim 21, wherein the first and second capacitor plates are at least partially exposed to the solution when the electroosmotic pump is activated.

23. The sensor module of claim 18, further comprising an electronics assembly, the electronics assembly comprising a first plurality of pins connecting the electronics assembly to the first capacitive plate, and a second plurality of pins connecting the electronics assembly to the second capacitive plate.

24. The sensor module of claim 23, wherein the controller is integrated into the electronics assembly.
